(19) United States
(12) Patent Application Publication
(10) Pub. No.: US 2026/0224734 A1
(43) Pub. Date: Aug. 6, 2026

(54) METHOD FOR PREPARING MULTIFUNCTIONAL IMMUNO-AGONIST FOR REPROGRAMMING TUMOR-ASSOCIATED MACROPHAGES

(71) Applicant: Beijing University of Chemical Technology, Beijing (CN)

(72) Inventors: Yongqin LV, Beijing (CN); Qingmei SONG, Beijing (CN)

(73) Assignee: Beijing University of Chemical Technology, Beijing (CN)

(21) Appl. No.: 19/460,361

(22) Filed: Jan. 27, 2026

(30) Foreign Application Priority Data
Jan. 31, 2025 (CN) 202510127177.6

(51) Int. Cl. A61K 47/69; A61K 47/62; B82Y 5/00; B82Y 25/00

(52) U.S. Cl. CPC A61K 47/6923; A61K 47/62; B82Y 5/00; B82Y 25/00

(57) ABSTRACT

A multifunctional immuno-agonist for reprogramming tumor-associated macrophages and a method for its preparation are provided. A solid-phase carrier is constructed by coupling a siglec-10 receptor epitope to the surface of thiolated Fe₃O₄ magnetic beads through cysteine modification using a disulfide bond as the linkage. An amino acid-derived monomer is synthesized and polymerized on the carrier surface to form a functional polymer layer. A thiol-based reducing agent is then applied to remove the epitope template, yielding a polymer layer containing a specific recognition cavity. The resulting immuno-agonist is capable of binding to the siglec-10 receptor and anchoring macrophages through the recognition cavity, and the magnetic component enables localized accumulation under an external magnetic field. The material can promote macrophage polarization and modulate their immune activity. The immuno-agonist is designed for applications involving tumor-associated macrophages.

### CROSS REFERENCE TO THE RELATED APPLICATIONS

This application is based upon and claims priority to Chinese Patent Application No. 202510127177.6, filed on Jan. 31, 2025, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBRZBC352_SequenceListing.xml, created on 01/21/2026, and is 1,799 bytes in size.

### TECHNICAL FIELD

The present invention relates to the field of biomedical technologies, and specifically to a method for preparing a multifunctional immuno-agonist for reprogramming tumor-associated macrophages.

### BACKGROUND

Macrophages are important components of the immune system and exhibit significant phenotypic diversity. In the tumor microenvironment, tumor tissues attract resident macrophages and circulating monocytes, making macrophages the major immune cell population. Macrophages are typically classified into pro-inflammatory (M1) and anti-inflammatory (M2) phenotypes. In the tumor microenvironment, especially tumor-associated macrophages (TAMs), macrophages tend to adopt an anti-inflammatory M2-like phenotype under various stimuli. This phenotype is characterized by upregulation of cytokines, proteases, and immunosuppressive molecules, thereby promoting tumor progression, metastasis, invasion, and resistance to cancer therapy. In many cancer types, increased TAM infiltration is generally associated with poor patient prognosis. Therefore, TAMs have become promising targets in cancer therapy.

Currently, various strategies have been proposed for targeting TAMs in cancer treatment. These include regulating the signaling pathways involved in macrophage recruitment to modulate monocyte migration into tumor sites. Strategies aimed at depleting TAMs and related approaches are also being developed and optimized. However, these approaches often overlook the inherent immune functions of macrophages. As a response, reprogramming TAMs has emerged as a strategy that activates pro-inflammatory signaling pathways or enhances their phagocytic activity toward tumor cells. Toll-like receptor agonists and cytokines have been widely used for inducing TAM repolarization.

Sialic acid-binding immunoglobulin-like lectin 10 (siglec-10) is an inhibitory receptor highly expressed on macrophages and plays a crucial role in immune regulation. Its high-affinity binding with tumor biomarker CD24 (primarily dependent on sialylation) highlights CD24 as a main ligand of siglec-10. Interaction between tumor cell-expressed CD24 and macrophage-expressed siglec-10 initiates an inhibitory signaling cascade mediated primarily by SHP-1 and/or SHP-2 phosphatases containing Src homology 2 domains. These phosphatases associate with immunoreceptor tyrosine-based inhibitory motifs (ITIMs) or ITIM-like motifs and the cytoplasmic tail of siglec-10, effectively blocking Toll-like receptor-mediated inflammatory responses and the cytoskeletal rearrangements required for macrophage-mediated phagocytosis. Consequently, macrophages fail to recognize tumor cells, enabling tumor immune escape. Notably, blocking the CD24/siglec-10 signaling axis has been shown to exhibit significant efficacy in suppressing tumors.

### SUMMARY

The objective of the present invention is to provide a method for preparing a multifunctional immuno-agonist capable of reprogramming tumor-associated macrophages. Using a siglec-10 receptor epitope as the target and a disulfide bond as the “bridge,” the invention innovatively develops a precise solid-phase-carrier-based preparation process.

Specifically, the target epitope is first conjugated to the surface of thiolated Fe₃O₄ magnetic beads via a cysteine residue at its terminus to construct a solid-phase carrier. Subsequently, derivatives of phenylalanine and tyrosine are designed and synthesized based on the epitope sequence and polymerized on the surface of the solid-phase carrier to form a functional polymer layer. Finally, thiol-containing reducing agents such as glutathione, dithiothreitol, or tris(2-carboxyethyl)phosphine are used as the eluent to cleave the disulfide bond and remove the bound target, leaving behind a polymer layer containing specific recognition cavities.

The resulting immuno-agonist can target the siglec-10 receptor on macrophages and exhibits multiple functions: (i) it enables efficient binding to macrophages in vivo through the specific recognition cavities in the polymer matrix; (ii) under an external magnetic field, the immuno-agonist rapidly accumulates in tumor regions, anchoring the associated macrophages at the tumor site and prolonging their retention time, thereby enhancing macrophage endogenous phagocytic capability; (iii) macrophages internalizing the immuno-agonist are effectively induced to polarize toward the M1 phenotype, further activating their anti-tumor immune functions. The invention thus provides an innovative therapeutic agent targeting TAMs, capable of enhancing macrophage anti-tumor activity while minimizing effects on normal tissues.

The preparation method includes:

Preparing a mixed reaction solution of N-isopropylacrylamide (NIPAm), a functional monomer, N,N′-methylenebisacrylamide (Bis), and N-tert-butylacrylamide (TBAm) at a concentration of 13-65 mM, wherein their molar ratios are (32-98):(1-45):(1-5):(1-20). Adding the solid-phase carrier, introducing 1-10 mM ammonium persulfate (APS) under nitrogen, and conducting an open-air polymerization at 38-45° C. for 8-16 hours followed by magnetic separation. Adding 10-20 mM thiol reducing agent and eluting at 0-8° C. for 12-24 hours to obtain the product.

The functional monomer is N-acryloyl-L-tyrosine (AcTyr) and/or N-acryloyl-L-phenylalanine (AcPhe).

The solid-phase carrier is a peptide conjugated to thiolated Fe₃O₄ magnetic beads.

The core amino acid sequence of the epitope peptide is CFRVERGSYVRYNFMND, shown in SEQ ID NO: 1.

The preparation of the solid-phase carrier includes dissolving the peptide in 0.01-0.5 M NH₄HCO₃ to form a 0.01-0.5 mM solution, adding thiolated Fe₃O₄ magnetic beads, stirring at 20-30° C. for 4-8 hours, and magnetically separating, repeated at least twice.

The thiolated Fe₃O₄ beads have a particle size of 40-80 nm.

The stirring speed is 650-1200 rpm.

The thiol reducing agent is selected from glutathione, dithiothreitol, and tris(2-carboxyethyl)phosphine.

### Beneficial Effects

(1) The invention achieves effective elution of the target via cleavable disulfide bonds, solving template-elution difficulties of the prior art and improving recognition efficiency. The immuno-agonist exhibits affinity K_{D} values for siglec-10 as low as 10⁻¹² M, nearly 1000-fold higher than that for control proteins (e.g., human serum albumin).
(2) The multifunctional immuno-agonist rapidly accumulates at tumor sites under a magnetic field, enabling precise localization to the tumor microenvironment. Fluorescence imaging verifies successful targeting of macrophages at tumor sites in mice.
(3) After uptake, the immuno-agonist promotes macrophage polarization toward the M1 phenotype and enhances anti-tumor immune effects. In vivo treatment experiments showed tumor growth inhibition>30% and >20% survival at 45 days.

### BRIEF DESCRIPTION OF THE DRAWINGS

FIGS. 1A-1B are NMR spectra of AcTyr (FIG. 1A) and AcPhe (FIG. 1).

FIGS. 2A-2B are flow cytometry results of immuno-agonist SA2 binding to RAW264.7 (FIG. 2A) and BMDC (FIG. 2B) cells.

FIGS. 3A-3B show phagocytosis of tumor cells by RAW264.7 macrophages pretreated with SA2 (FIG. 3A) or mAb (FIG. 3B).

FIG. 4 shows in vivo fluorescence imaging in mice.

FIGS. 5A-5C show tumor size, survival rate, and body-weight change after treatment.

### DETAILED DESCRIPTION OF THE EMBODIMENTS

### Example 1

### 1. Preparation of the Solid-Phase Carrier

30 mg of thiolated Fe₃O₄ magnetic beads (particle size 50 nm) were added into a 0.1 mM peptide solution (amino acid sequence CFRVERGSYVRYNFMND, shown in SEQ ID NO: 1), wherein the peptide solution was prepared using a 0.1 M NH₄HCO₃ buffer solution. The mixture was stirred at room temperature at 800 rpm for 4 hours, followed by magnetic separation to remove the solution. The above operation was repeated three times to ensure that the peptide was fully bound to the surface of the magnetic beads.

### 2. Synthesis of the Functional Monomer N-Acryloyl-L-Tyrosine (AcTyr)

L-tyrosine (10 mmol) was dissolved in 8 mL of an aqueous NaOH solution (3.25 M). Under uniform stirring, acryloyl chloride (10 mmol) was added dropwise to the solution. The resulting mixture was stirred in an ice-water bath for 1 hour and then further stirred at room temperature for 6 hours. The reaction mixture was extracted three times with 20 mL of ethyl acetate. The organic phase was washed twice with 20 mL of 0.1 M HCl aqueous solution, followed by drying, filtration, and concentration using a rotary evaporator to obtain a white, fluffy solid product.

¹HNMR (400 MHz, DMSO-d₆): δ 5.6, 6.0 (—CH₂), 6.25 (—CH), 8.3 (—NH), 4.4 (—CH), 12.6 (—COOH), 2.9 (—CH₂), 6.6, 7.0 (—C₆H₄), 9.2 (—OH).

### 3. Synthesis of the Functional Monomer N-Acryloyl-L-Phenylalanine (AcPhe)

L-phenylalanine (0.020 mol) was dissolved in 20 mL of a 2 M sodium hydroxide aqueous solution and stirred until homogeneous. Subsequently, acryloyl chloride (0.022 mol) was added dropwise to the mixed solution of L-phenylalanine and sodium hydroxide. During the addition of acryloyl chloride, the reaction mixture was maintained below 0° C. by external cooling using an ice bath. The reaction was then continued by stirring at room temperature for 2 hours. Finally, the product was collected by vacuum filtration, washed, and dried to obtain a white solid sample.

¹H NMR (400 MHz, DMSO-d₆): δ 5.6, 6.0 (—CH₂), 6.25 (—CH), 8.3 (—NH), 4.5 (—CH), 2.9, 3.1 (—CH₂), 6.6, 7.25 (—C₆H₄).

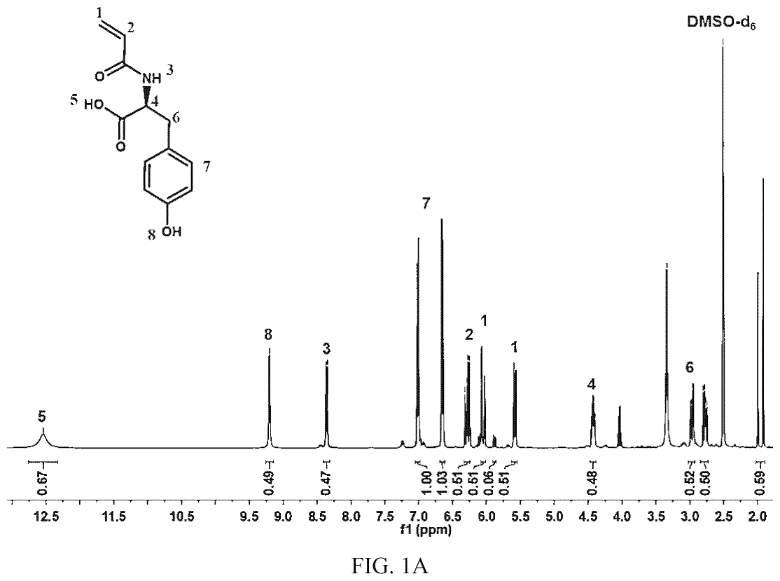

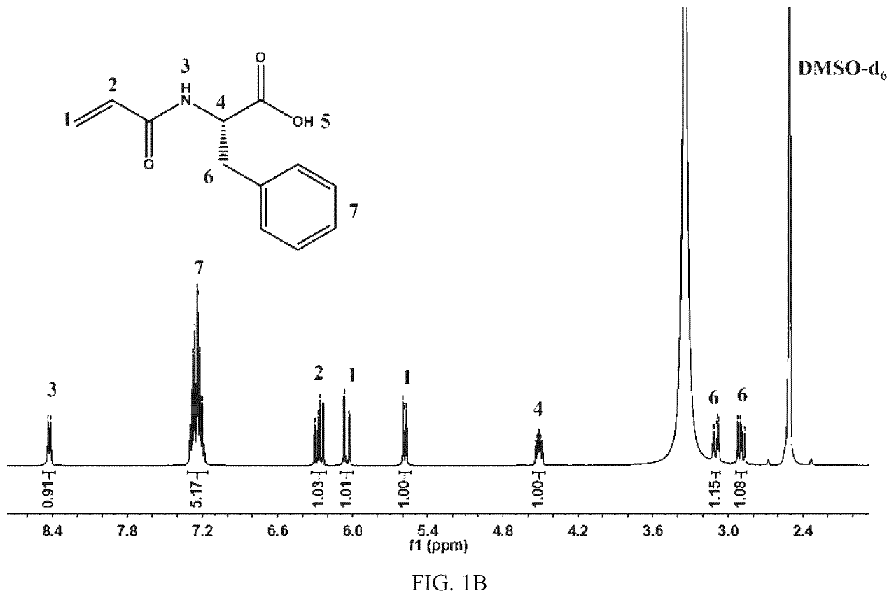

FIGS. 1A-1B show the NMR spectra of the synthesized N-acryloyl-L-tyrosine (AcTyr) (FIG. 1A) and N-acryloyl-L-phenylalanine (AcPhe) (FIG. 1i), demonstrating that pure AcTyr and AcPhe were successfully synthesized.

### Example 1: Synthesis of the Immuno-Agonist (SA1)

A mixed reaction solution was prepared by dissolving 1.02 mmol of NIPAm, 0.3 mmol of AcTyr (dissolved in 1 mL of 10 mM NaOH aqueous solution), 0.03 mmol of Bis, and 0.075 mmol of TBAm in water, followed by the addition of 30 mg of the solid-phase carrier. The final reaction volume was adjusted to 50 mL. Under continuous nitrogen flow, 0.26 mmol of APS was added, and the mixture was subjected to an open-air stirring polymerization reaction at 40° C. for 12 hours, after which magnetic separation was performed to remove the solution. After the reaction was completed, the supernatant was removed by magnetic separation. The resulting nanoparticle suspension was transferred to a 4° C. refrigerator, and 10 mM glutathione aqueous solution was added and allowed to stand overnight. SA1 was collected for later use.

By adding 2 mg of rhodamine B (dissolved in 1 mL of water) into the above pre-polymerization monomer solution while keeping the other reaction conditions unchanged, rhodamine-B-labeled SA1 could be obtained.

### Example 2: Synthesis of the Immuno-Agonist (SA2)

A mixed reaction solution was prepared by dissolving 0.505 mmol of NIPAm, 0.065 mmol of AcPhe, 0.013 mmol of Bis, and 0.033 mmol of TBAm in water, followed by the addition of 30 mg of the solid-phase carrier. The final reaction volume was adjusted to 50 mL. Under continuous nitrogen flow, 0.26 mmol of APS was added, and the mixture was subjected to an open-air stirring polymerization reaction at 40° C. for 12 hours, after which magnetic separation was performed to remove the solution. After the reaction was completed, the supernatant was removed by magnetic separation. The resulting nanoparticle suspension was transferred to a 4° C. refrigerator, and 10 mM glutathione aqueous solution was added and allowed to stand overnight. SA2 was collected for later use.

By adding 2 mg of rhodamine B (dissolved in 1 mL of water) into the above pre-polymerization monomer solution while keeping the other reaction conditions unchanged, rhodamine-B-labeled SA2 could be obtained.

### 4. Affinity of the Immuno-Agonists for the Siglec-10 Protein Receptor

The binding K_{D} values of the immuno-agonists SA1 and SA2 with the siglec-10 protein were measured using bio-layer interferometry (BLI). As shown in Table 1, the affinity K_{D} values of SA1 and SA2 for the siglec-10 protein can reach 10⁻¹¹ M or lower.

TABLE 1
BLI Results of Immuno-Agonists Binding to the siglec-10 Protein

| | K_{D} (M) | kₒₙ (1/Ms) | k_{dis} (1/s) | Full (R²) |
|---|---|---|---|---|
| SA₁ | 7.3 × 10⁻¹² | 4.14 × 10⁸ | 3.02 × 10⁻³ | 0.9675 |
| SA₂ | 4.81 × 10⁻¹¹ | 6.29 × 10⁷ | 3.03 × 10⁻³ | 0.9308 |

Selectivity of SA1 and SA2: The selectivity of the immuno-agonists SA1 and SA2 was evaluated using five control proteins: human serum albumin (HSA, pI=4.6), 7-globulin (Glo, pI=6.9), fibrinogen (Fib, pI=5.5), myoglobin (Myo, pI=7.0), and ribonuclease A (RNA, pI=8.9). The affinity K_{D} values of SA1 and SA2 toward these five proteins were measured using bio-layer interferometry (BLI). The results are shown in Table 2 and Table 3. The binding values of SA2 toward these proteins were in the range of 10⁻⁸ to 10⁻⁹ M, which is approximately 1000-fold different from its affinity for siglec-10. In contrast, the binding values of SA1 toward these proteins were in the range of 10⁻⁹ to 10⁻¹¹ M. These findings indicate that SA2 exhibits superior binding affinity for siglec-10, promoted by electrostatic interactions, hydrogen bonding interactions, and hydrophobic interactions.

TABLE 2
BLI Results of Immuno-Agonist SA1 Binding to Control Proteins

| | K_{D} (M) | kₒₙ (1/Ms) | k_{dis} (1/s) | Full (R²) |
|---|---|---|---|---|
| HSA | 1.25 × 10⁻¹⁰ | 3.16 × 10⁷ | 3.97 × 10⁻³ | 0.9513 |
| Glo | 7.07 × 10⁻¹⁰ | 3.26 × 10⁶ | 2.3 × 10⁻³ | 0.9673 |
| Fib | 6.99 × 10⁻¹¹ | 2.8 × 10⁷ | 1.96 × 10⁻³ | 0.7484 |
| Myo | 1.43 × 10⁻⁹ | 1.64 × 10⁶ | 2.35 × 10⁻³ | 0.9429 |
| RNA | 2.92 × 10⁻¹⁰ | 4.55 × 10⁶ | 1.33 × 10⁻³ | 0.5829 |

TABLE 3
BLI Results of Immuno-Agonist SA2 Binding to Control Proteins

| | K_{D} (M) | kₒₙ (1/Ms) | k_{dis} (1/s) | Full (R²) |
|---|---|---|---|---|
| HSA | 3.21 × 10⁻⁹ | 6.98 × 10⁵ | 2.24 × 10⁻³ | 0.9389 |
| Glo | 7.87 × 10⁻⁹ | 2.38 × 10⁵ | 1.87 × 10⁻³ | 0.8507 |
| Fib | 1.62 × 10⁻⁸ | 1.67 × 10⁵ | 2.71 × 10⁻³ | 0.7941 |
| Myo | 1.98 × 10⁻⁸ | 1.4 × 10⁵ | 2.77 × 10⁻³ | 0.8539 |
| RNA | 2.08 × 10⁻⁸ | 1.35 × 10⁵ | 2.8 × 10⁻³ | 0.8194 |

### 5. In Vitro Cell Experiments

### 5.1 Binding of SA2 to Cells

Mouse macrophage RAW264.7 cells were selected as siglec-10 receptor-positive cells, and mouse dendritic cells (BMDCs) were selected as siglec-10 receptor-negative cells. In the flow cytometry experiment, rhodamine-B-labeled SA2 dispersions (120 μg/mL) were used to stain RAW264.7 cells for 1 hour, with untreated cells serving as the control. To confirm siglec-10-positive expression, the cells were also stained with PE-labeled anti-siglec-10 antibodies for 1 hour. The cells were then washed, digested, and collected for flow cytometry analysis.

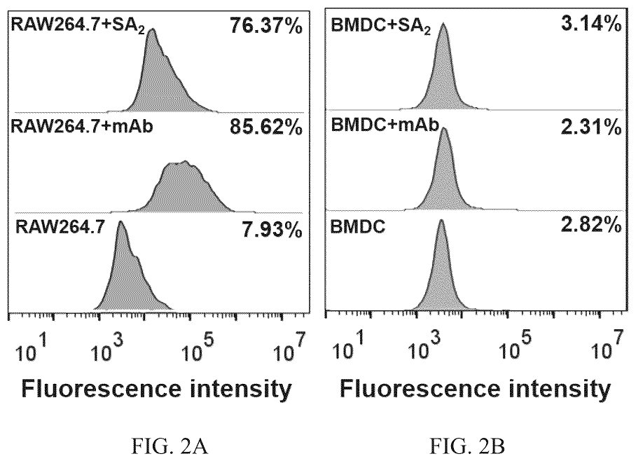

As shown in the flow cytometry results in FIGS. 2A-2B, the positive binding rate of SA2 with RAW264.7 cells was 76.37%, which is close to the binding rate of commercial protein antibodies (mAb) with RAW264.7 cells (85.62%). In contrast, SA2 exhibited almost no binding to siglec-10-negative BMDC cells, with a positive binding rate of only 3.14%.

### 5.2 In Vitro Phagocytosis of Tumor Cells by RAW264.7 Macrophages

Mouse ovarian cancer ID8 tumor cells were labeled with GFP. The macrophage RAW264.7 cells were labeled with the pHRodoRed dye. pHRodoRed is a pH-sensitive fluorescent dye that generates red fluorescence in the acidic environment of lysosomes when macrophages phagocytose tumor cells. After incubation of macrophages with SA2 (120 μg/mL) or sterile phosphate-buffered saline (PBS, pH=7.4) for 1 hour, the cells were washed. The pretreated macrophages were then digested and co-cultured with tumor cells (10⁵ cells per dish) at a ratio of 2:1 at 37° C. After 12 h, 24 h, and 36 h of co-culture, the cells were collected, washed, and analyzed by flow cytometry.

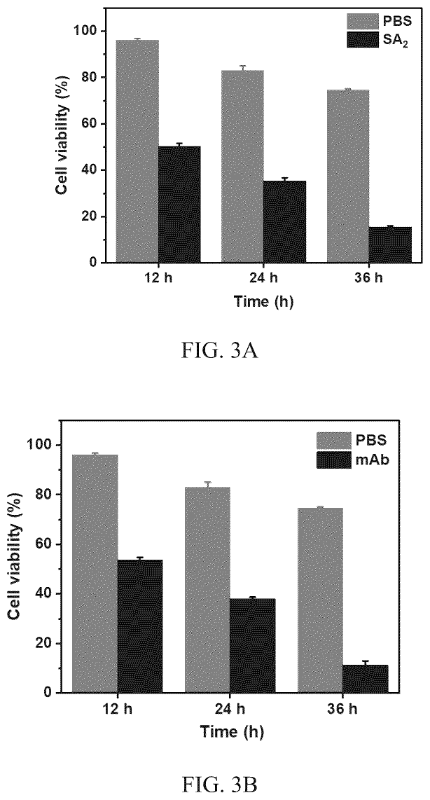

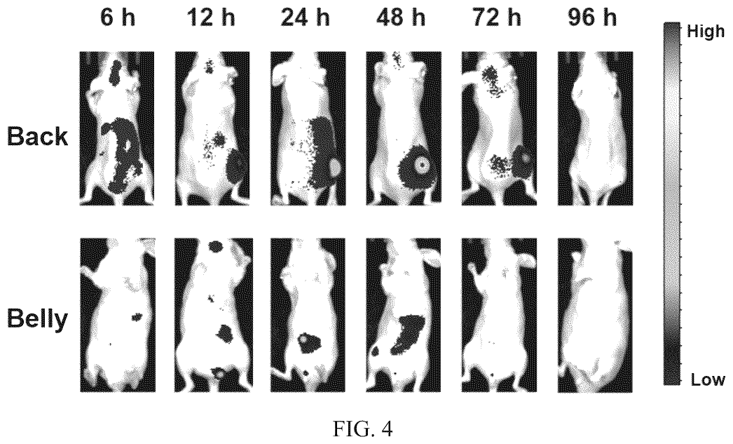

As shown in FIG. 3A, after 12 hours of co-incubation of GFP-labeled ID8 mouse ovarian cancer cells with SA2-pretreated RAW264.7 macrophages, the macrophages exhibited significant phagocytic activity toward the tumor cells, with a phagocytosis rate of approximately 50%. In contrast, PBS-treated macrophages did not display noticeable tumor-phagocytosis activity. This indicates that SA2 significantly enhances the ability of macrophages to clear tumor cells by blocking tumor immune escape signals. Furthermore, as the co-culture time increased to 24 hours, the clearance rate of tumor cells by SA2-pretreated macrophages remained around 35%. When the co-culture time reached 36 hours, the tumor cell survival rate sharply dropped to below 20%. Notably, comparison with the phagocytic activity of the monoclonal antibody (mAb)-treated group (FIG. 3B) showed that the synthetic SA2 achieved an effect comparable to that of the antibody.

### 6. In Vivo Targeting of the Tumor Microenvironment by the Immuno-Agonist SA2

### 6.1 Synthesis of Cy7-Fluorescently Labeled Poly(N-Acryloyl-L-Phenylalanine) Polymer (SA2@Cy7)

A mixed reaction solution was prepared by dissolving 0.505 mmol of NIPAm, 0.065 mmol of AcPhe, 0.013 mmol of Bis, and 0.033 mmol of TBAm in water, followed by the addition of 30 mg of the solid-phase carrier and 0.0065 mmol of APM. The final reaction volume was adjusted to 50 mL. Under continuous nitrogen flow, 0.26 mmol of APS was added, and the mixture was subjected to an open-air stirring polymerization reaction at 40° C. for 12 hours, after which magnetic separation was performed to remove the solution. After the reaction was completed, the supernatant was removed by magnetic separation. The resulting nanoparticle suspension was transferred to a 4° C. refrigerator, and 10 mM glutathione aqueous solution was added and allowed to stand overnight. The amino-modified SA2 was collected for later use.

Cy7-NHS fluorescent dye and the amino-modified SA2 were mixed at equimolar concentrations in phosphate-buffered saline (PBS, pH=7.4) and reacted overnight at room temperature. After the reaction, the resulting Cy7-labeled nanoparticle solution was transferred to a dialysis bag (molecular weight cutoff. 1000 Da) and dialyzed for 3 days. The Cy7-labeled SA2 (SA2@Cy7) was collected for later use.

### 6.2 In Vivo Fluorescence Imaging in Mice

A syngeneic ID8 ovarian cancer mouse model was established by subcutaneously injecting 10⁷ cells into the right hind leg of healthy 4-week-old nude mice. When the tumor volume reached approximately 100 mm³, the experiment was conducted. The synthesized SA2@Cy7 was filtered through a sterile 0.2-μm membrane and adjusted to a concentration of 0.5 mg/mL. Mice (n=3 per group) were used to evaluate the temporal in vivo distribution of SA2@Cy7. Approximately 200 μL of SA2@Cy7 was administered via tail vein injection, and an external magnetic field was simultaneously applied to the tumor site of the mice. Fluorescence imaging was performed at several time points after administration: 6 h, 12 h, 24 h, 48 h, 72 h, and 96 h. Whole-body fluorescence signals were monitored non-invasively over a period of four days to track the circulation and biodistribution kinetics of the nanoparticles.

As shown in FIG. 4, 12 hours after intravenous injection, SA2@Cy7 accumulated within the tumor microenvironment, indicating that SA2@Cy7 successfully targeted macrophages at the tumor site in mice under external magnetic field intervention, thereby achieving precise localization to the tumor microenvironment.

7. In Vivo Tumor Treatment Experiment with the Immuno-Agonist SA2

Aright-flank tumor model was established using 4-week-old female BALB/c mice. First, 1×10⁷ ID8 cells were subcutaneously injected into the right thigh of the mice to establish the tumor model. The mice were then randomly divided into three groups, with five mice in each group. When the tumor volume reached 50 mm³, treatment was initiated. The treatment was administered once every three days, for a total of four treatments. The first group received intravenous injection of PBS as the control group. The second group received intravenous injection of the SA2 dispersion (dose: 5 mg/kg). The third group received intravenous injection of mAb (dose: 100 kg). Tumor volume, changes in mouse body weight, and survival rate were recorded.

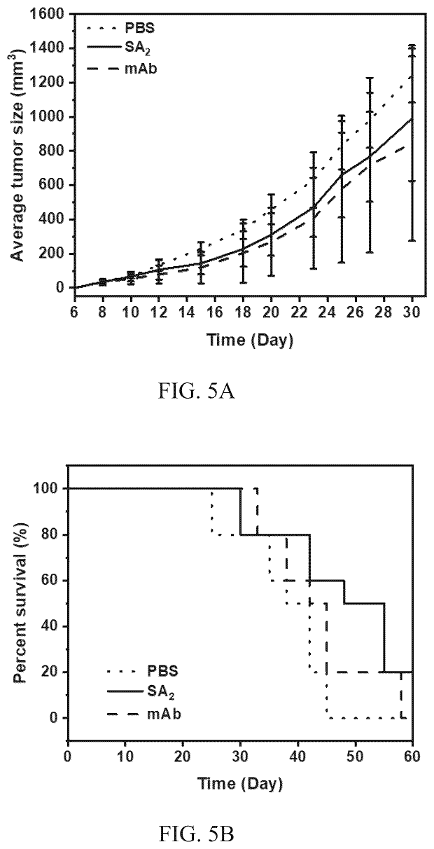

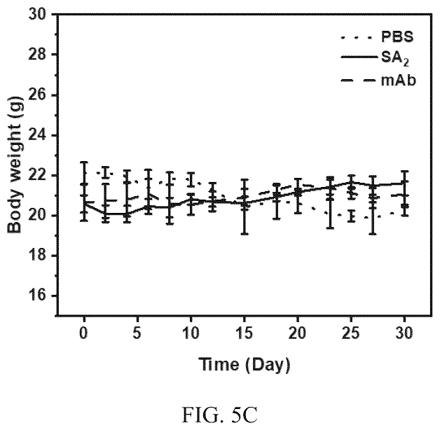

As shown in FIGS. 5A-5C, compared with PBS, SA2 inhibited tumor growth and extended the survival of the mice, with a survival rate of 20% at 45 days. This indicates that SA2 exhibits effective anti-tumor therapeutic activity. Furthermore, comparison with the therapeutic effect of mAb in vivo shows that SA2 achieved results similar to mAb in inhibiting tumor growth and prolonging survival time, demonstrating that the tumor-cell-killing capability of the synthetic SA2 is fully comparable to that of commercial biological antibodies.

What is claimed is:

1. A method for preparing a multifunctional immuno-agonist for reprogramming tumor-associated macrophages, comprising: preparing a mixed reaction solution of N-isopropylacrylamide, a functional monomer, N,N′-methylenebisacrylamide, and N-tert-butylacrylamide at a concentration of 13-65 mM, wherein a molar ratio of the N-isopropylacrylamide, the functional monomer, the N,N′-methylenebisacrylamide, and the N-tert-butylacrylamide is (32-98):(1-45):(1-5):(1-20); adding a solid-phase carrier; under continuous nitrogen flow, adding 1-10 mM ammonium persulfate, and performing an open-air stirring polymerization reaction at 38-45° C. for 8-16 hours, followed by magnetic separation to remove a first supernatant to obtain remaining nanoparticles; and finally adding a 10-20 mM thiol reducing agent solution to the remaining nanoparticles and performing elution at 0-8° C. for 12-24 hours to obtain the multifunctional immuno-agonist as a product.

2. The method according to claim 1, wherein the functional monomer is N-acryloyl-L-tyrosine and/or N-acryloyl-L-phenylalanine.

3. The method according to claim 1, wherein the solid-phase carrier is a peptide coupled to thiolated Fe₃O₄ magnetic beads.

4. The method according to claim 3, wherein the core amino acid sequence of the peptide is CFRVERGSYVRYNFMND, shown in SEQ ID NO: 1.

5. The method according to claim 3, wherein a preparation of the solid-phase carrier comprises: dissolving the peptide in 0.01-0.5 M NH₄HCO₃ solution to form a 0.01-0.5 mM solution, adding the thiolated Fe₃O₄ magnetic beads to obtain a mixture, stirring the mixture at 20-30° C. for 4-8 hours, and magnetically separating magnetic beads to remove a second supernatant, wherein steps of the adding, the stirring, and the magnetically separating are repeated at least twice.

6. The method according to claim 5, wherein particle size of the thiolated Fe₃O₄ magnetic beads is ranging from 40 nm to 80 nm.

7. The method according to claim 5, wherein a stirring speed is 650-1200 rpm.

8. The method according to claim 1, wherein a thiol reducing agent in the 10-20 mM thiol reducing agent solution is one or more selected from glutathione, dithiothreitol, and tris(2-carboxyethyl)phosphine.


SEQUENCE LISTING (1 sequence)

SEQ ID NO: 1 - protein, 17 aa, synthetic construct
CFRVERGSYVRYNFMND